(19) United States
(12) Patent Application Publication
(10) Pub. No.: US 2021/0369102 A1
(43) Pub. Date: Dec. 2, 2021

(54) REMOTE/AT-HOME VISION TESTING SYSTEM AND METHOD

(71) Applicant: The Hilsinger Company Parent LLC, Plainville, MA (US)

(72) Inventors: Kevin A. Butler, Downers Grove, IL (US); Michael Spencer, Hoffman Estates, IL (US); Joseph A. Marino, Alpine, WY (US)

(73) Assignee: THE HILSINGER COMPANY

(21) Appl. No.: 17/333,513

(22) Filed: May 28, 2021

Related U.S. Application Data
(60) Provisional application No. 63/031,297, filed on May 28, 2020.

(51) Int. Cl. A61B 3/032; G06T 3/40; A61B 5/00; G01B 21/16

(52) U.S. Cl. CPC A61B 3/032; G06T 3/40; G01B 21/16; A61B 5/742; A61B 5/7405

(57) ABSTRACT

A vision testing system is provided that tests the vision of an associated patient. A display of the vision testing unit includes a first surface for displaying a vision test thereon. A processing unit is operatively associated with the display and selectively displays optotypes on the first surface. A monitoring system senses a distance between the associated patient and the display, and operationally interfaces with the processing unit to provide data representative of the distance of the associated patient from the display. A cue may be provided to the associated patient in response to the sensed distance. In addition, ambient lighting conditions may be sensed at the display and brightness of the display altered in response to the ambient light.

This application claims the priority benefit of and expressly incorporates by reference U.S. provisional application Ser. No. 63/031,297, filed May 28, 2020.

### BACKGROUND

This invention relates to a vision testing system, and more particularly for modifying existing vision testing systems and associated methods that allow remote use of the system, such as at home.

Vision testing typically occurs at the office or facility of an optometrist, ophthalmologist, clinic, etc., under carefully controlled conditions. Vision testing systems are set up in specialized rooms that control, among other things, required testing parameters such as physical distance between the patient and a display surface such as a monitor. The system display or monitor can be an LED (light-emitting diode), LCD (liquid crystal display), CRT (cathode ray tube), illuminated surface, etc., and is situated a certain, predetermined distance from a patient so that careful control is maintained over calibrated size and distance of the optotypes (optical characters such as letters, numbers, etc.) displayed on the display surface relative to the location of the patient from the display surface. In addition, ambient light in the test room is carefully controlled. Although lux may vary from one test room to another, the ambient room lighting is controlled/checked (e.g., typically set for screen luminance at 85 candelas per meter squared (cd/m²) and most trials are conducted at less than 15 lux for the room; so confirming that the room is dark and the display may be adjusted to meet the desired screen luminance of 85 cd/m² (or whatever the desired level is for a given test/study), and the screen brightness of the test monitor is typically adjusted automatically in response to the ambient lighting. This assures ideal test conditions and eliminates undesired variability in the testing and test results. Exemplary vision test systems include, for example, commonly owned U.S. Pat. No. 10,244,938 (Vision Testing System and Method); U.S. Pat. No. 10,182,713 (Glare Assembly For Eye Test Display); U.S. Pat. No. 8,425,040 (Astigmatic Axis Independent. Spatial Frequency And Contrast Sensitivity Target And Method); and U.S. Published patent applications 200810143961A1 (Apparatus And Method For Testing Visual Acuity And Fixation Control); and 2004/0141152A1 Apparatus And Method For Conducting Vision Screening); the disclosures of which are expressly incorporated herein by reference.

The recent pandemic, however, and the associated lockdown have closed such facilities and inconvenienced both the healthcare professional and patient, as well as resulting in undesired delay in vision testing.

A need exists for an improved system that provides at least one or more of the above-described features, as well as still other features and benefits.

### SUMMARY

A vision testing system is modified to allow remote or at home vision testing to be conducted while maintaining desired control over testing parameters.

The testing system includes a first monitoring feature that senses the physical distance between the patient and a display surface such as a monitor on which the optotypes are displayed.

If the patient is not located at the proper distance from the display monitor, the first monitoring feature can (i) prompt the patient to move closer to or further away from the display monitor, or (ii) dynamically alter the test display to re-size the optotypes, or (iii) use a combination of prompts or dynamic alterations as needed.

The testing system also includes a second monitoring feature that senses ambient lighting in the area where the testing occurs.

The second monitoring feature can (i) provide a prompt that ambient lighting must be adjusted, or (ii) turn off ambient lighting and/or dynamically alter the display brightness; or (iii) use a combination of prompts and lighting alterations as needed.

The first and/or second monitoring features can be incorporated into new testing systems or provided as aftermarket, add-on features to adapt existing systems to allow remote or at-home vision testing.

Substantially all vision tests are distance based, i.e. calibration of optotypes for size and distance from the patient, except perhaps color testing, so that modification of existing systems to include these features or the provision of new vision testing systems that include these features is highly desirable.

The first and/or second monitoring features may be used for set up of the vision test, i.e. prior to initiation of the vision test, and can also be advantageously used for continuous or periodic monitoring during the vision test.

The first and/or second monitoring features may employ a wide variety of prompts that may include audible and/or visible signals or cues. The first and/or second monitoring features may also include the ability to alter one or more parameters of the vision test system, e.g., adjust the optotype size, adjust the brightness of the display surface, turn off an ambient light source, etc., either prior to and/or during the vision test.

The test data, including information relating to the first and/or second monitoring features, can be stored locally (e.g., in the local memory of a computer such as a laptop) and then the information streamed or periodically uploaded to the remote location when remote communication is available.

If the features being monitored (distance between patient and display monitor, and ambient lighting and/or screen brightness) fall outside the desired operating parameters, the vision test can be paused and subsequently resumed when the operating parameters are met, or the vision test stopped, or the vision test started anew.

The data relating to the first and/or second monitoring features may include video recording of test set-up, or video recording of both set-up and the vision test, for example using a web camera. The video recordings can be included as part of the stored and/or uploaded data associated with a vision test.

Benefits and advantages of the present disclosure will become more apparent from reading and understanding the following detailed description.

### BRIEF DESCRIPTION OF THE DRAWINGS

FIG. 1 is a schematic representation of the new vision testing system.

FIG. 2 is a flowchart illustrating representative process steps and decisions to be made in association with the new vision testing system.

### DETAILED DESCRIPTION

The following description with reference to the accompanying drawings is provided to assist in a comprehensive understanding of one or more embodiments of the present disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the present disclosure. Various exemplary embodiments of the present disclosure are not limited to the specific details of different embodiments and should be construed as including all changes and/or equivalents or substitutes included in the ideas and technological scope of the appended claims. In describing the drawings, where possible similar reference numerals are used for similar elements.

The terms “include” or “may include” used in the present disclosure indicate the presence of disclosed corresponding functions, operations, elements, and the like, and do not limit additional one or more functions, operations, elements, and the like. In addition, it should be understood that the terms “include”, “including”, “have” or “having” used in the present disclosure are to indicate the presence of components, features, numbers, steps, operations, elements, parts, or a combination thereof described in the specification, and do not preclude the presence or addition of one or more other features, numbers, steps, operations, elements, parts, or a combination thereof.

The terms “or” or “at least one of A or/and B” used in the present disclosure include any and all combinations of words enumerated with them. For example, “A or B” or “at least one of A or/and B” mean including A, including B, or including both A and B.

Although the terms such as “first” and “second” used in the present disclosure may modify various elements of the different exemplary embodiments, these terms do not limit the corresponding elements. For example, these terms do not limit an order and/or importance of the corresponding elements, nor do these terms preclude additional elements (e.g., second, third, etc.) The terms may be used to distinguish one element from another element. For example, a first mechanical device and a second mechanical device all indicate mechanical devices and may indicate different types of mechanical devices or the same type of mechanical device. For example, a first element may be named a second element without departing from the scope of the various exemplary embodiments of the present disclosure, and similarly, a second element may be named a first element.

It will be understood that, when an element is mentioned as being “connected” or “coupled” to another element, the element may be directly connected or coupled to another element, and there may be an intervening element between the element and another element. To the contrary, it will be understood that, when an element is mentioned as being “directly connected” or “directly coupled” to another element, there is no intervening element between the element and another element.

The terms used in the various exemplary embodiments of the present disclosure are for the purpose of describing specific exemplary embodiments only and are not intended to limit various exemplary embodiments of the present disclosure. As used herein, the singular forms are intended to include the plural forms as well, unless the context clearly indicates otherwise.

All of the terms used herein including technical or scientific terms have the same meanings as those generally understood by an ordinary skilled person in the related art unless they are defined otherwise. The terms defined in a generally used dictionary should be interpreted as having the same meanings as the contextual meanings of the relevant technology and should not be interpreted as having inconsistent or exaggerated meanings unless they are clearly defined in the various exemplary embodiments.

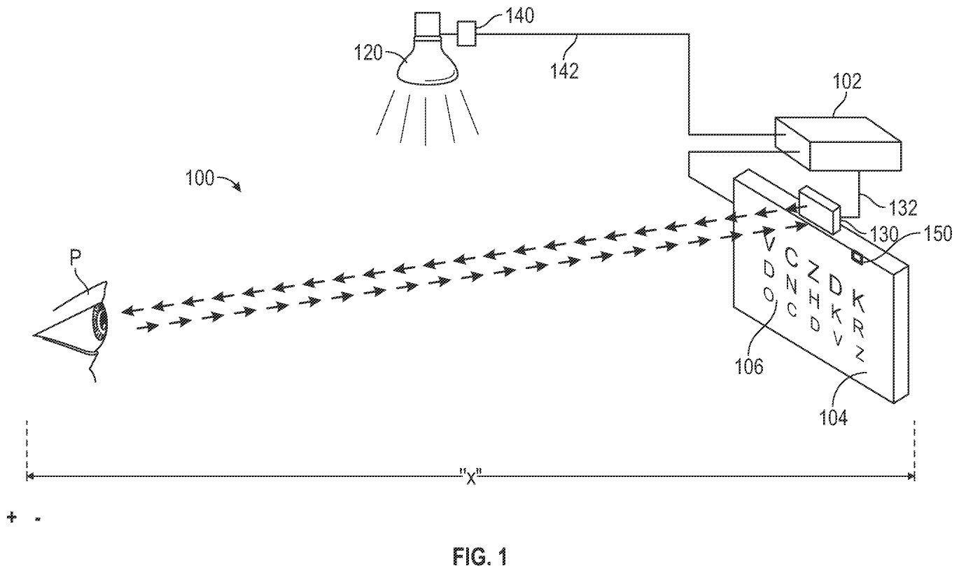

Turning to FIG. 1, there is shown a vision testing system 100 that includes a computer or central processing unit (CPU) 102 connected to a display surface such as monitor 104 on which optotypes 106 (e.g., letters, numbers, characters, shapes) are selectively displayed. The computer 102 runs or operates software that is either resident in memory of the computer, on a disk, memory stick, etc., or the computer is connected (e.g. wired or wirelessly) to a remote server or memory where the software resides. The software provides the commands for selectively displaying the optotypes 106 on the monitor 104 in a particular way. A patient P is positioned a predetermined distance “x” from the monitor 104. The size of the optotypes 106 displayed on the monitor 104 is determined by the distance “x” at which the patient P is positioned from the monitor. Typically, in an examination room of a healthcare professional, the system is set up in advance so that the patient P is located at the desired distance “x” from the monitor 104.

Ambient lighting is generally represented at 120. Again, ambient lighting 120 is typically set up in advance and controlled so that the patient P is positioned at the desired location “x” and under the desired conditions of the ambient lighting 120 to optimize the test results. The screen brightness of the monitor 104 and size of the optotypes 106 on the monitor are also controlled so that in conjunction with the ambient lighting 120 and distance “x”, ideal conditions for conducting the vision test are available or can be established prior to initiation of the vision test.

As will be appreciated, one or more of these parameters is difficult to establish and/or control if the system 100 is intended to be used remotely, for example at home. With continued reference to FIG. 1, the system 100 described so far is modified with one or more of the following features. The distance “x” is established or controlled by use of a first monitoring apparatus or feature 130 that senses the physical distance “x” between the monitor 104 on which the optotypes 106 are displayed and the patient P. Particularly, the first monitoring apparatus 130 is a distance sensing/measuring device that transmits a signal such as a light signal (e.g., laser), ultrasonic signal, or sound signal 132 preferably from a perimeter face of the monitor 104 toward the patient P. The patient P is oriented in a direction perpendicular to a plane defined by the screen or face of the monitor 104. With the patient P facing the monitor, the signal is transmitted or directed away from the monitor by a transmitter portion of the distance sensing device 130 and the signal will reflect off the patient and is thereby re-directed toward and received by a receiver portion of the distance sensing device. By measuring the time for the signal to travel from a transmitter portion of the distance measuring device to a receiver portion of the distance measuring device, the distance of the patient P from the monitor 104 can be calculated by the processing unit based on the measured time. The calculated distance is then compared with the ideal distance “x”, and the measured, calculated distance can either be increased (+) to reach the desired distance or the measured, calculated distance can be decreased (−) to reach the desired distance “x”. If the patient P is not located at the proper distance “x” from the display monitor 104, the first monitoring apparatus can prompt the patient to move closer to or further away from the display monitor. For example, an audible signal or cue and/or visual display or cue on the screen of the monitor 104 will direct the patient P to either increase or decrease the distance until the measured, calculated distance matches the desired proper distance “x”.

Alternatively, the measured, calculated distance can be used and input to the vision test software as represented by reference line 132 such that the software dynamically adjusts the sizes of the optotypes 106 to compensate for the difference between the measured, calculated distance and the desired, proper distance “x”.

Still another possibility is to use a combination of (i) prompting the patient P to increase or decrease the distance from the monitor and (ii) dynamically altering the test display to re-size the optotypes 106 on the monitor 104 as needed. Under one scenario, and without deemed to be a limiting example, the patient P may be prompted to either increase or decrease the distance from the monitor until the desired, proper distance “x” is achieved. If after a few iterations it becomes evident that the desired, proper distance “x” cannot be achieved, the prompting to increase or decrease the distance can cease and then the optotypes 106 will be re-sized or recalibrated in order to properly conduct the test.

When testing vision, the size of the optotype (letter, number, or character/target) 106 that the patient P is viewing during the eye examination is sized specifically to a given distance. This means that if a patient P moves closer or further from the display/monitor 104 of the vision test, the patient makes the test easier or more difficult which can impact the measurement and assessment of the vision of the patient. This invention advantageously uses the distant sensing device 130 to measure the distance from an object (patient. P) at some distance in front of the display monitor 104. As will be appreciated, this distant sensing device 130 can be one of various types such as a laser or other optical-based sensor, ultrasonic signal sensor, or a sonar-type sensor. Because the sensor apparatus 130 is constantly measuring the distance, the vision test can be sized specifically to the measured/calculated distance real-time, making the optotypes 106 smaller if the patient P moves closer or larger if the patient moves back. This allows consistency in the test making for more accurate and repeatable results.

The first sensor apparatus 130 sends out (transmits or emits from a transmitter portion of the sensor) a signal and then waits to receive (receiver portion of the sensor) the reflection. When the first sensor apparatus 130 receives the reflected signal, the sensor apparatus and/or computer calculates the time for the transmitted signal to be received, and uses the time to determine the distance of the object (patient P) from the first sensor apparatus. Using serial communication such as transistor to transistor logic (sometimes referred to as TTL serial) is easy to pair with a microcontroller since it operates in a voltage supply range of a microcontroller. The sensor 130 reports the distance in millimeters. Vision testing software connected to the sensor via the serial-TTL connection reads this constant stream of data and at given intervals, re-sizes the vision test based on the newly reported distance.

This can also be used in situations where an exact distance is critical. If a test needs to take place at 4 meters, for example, the data from the first sensor apparatus 130 can be used by the vision testing software to prompt the user/patient P to move away or closer until the distance “x” is reached. The software can then utilize the data from the first sensor apparatus 130 to prompt the patient. P to move based on the stream of the distance data. There may be situations where there could be (i) re-sizing of the vision test and/or (ii) prompting the patient P to move forward or backward depending on the measured, calculated distance versus the required distance “x” as noted above.

The modified remote testing system 100 of the present disclosure may also include a second monitoring apparatus or feature 140 that senses ambient lighting in the area where the testing occurs. In one preferred manner, the second sensing apparatus 140 inputs the measured lux as represented by reference line 142 to the computer 102. The second monitoring feature 140 can (i) dynamically alter the display brightness of the monitor 104, or (ii) provide a prompt that ambient lighting 120 must be adjusted (e.g., turned off); or (iii) use a combination of prompts and alterations as needed.

The first and/or second monitoring features 130, 140 can be incorporated into new vision testing systems 100 or provided as aftermarket, add-on features to adapt existing vision testing systems to allow remote or at-home vision testing. The first and/or second monitoring features 130, 140 may also be used for set-up of the vision test, i.e. prior to initiation of the vision test, and can also be advantageously used for continuous or periodic monitoring during the vision test.

The first and/or second monitoring features 130, 140 may employ a wide variety of prompts that may include audible and/or visible signals or cues, and/or as noted above communicate with the processing unit to alter one or more parameters of the vision test (e.g., ambient lighting, size of optotypes, screen brightness, etc.). Information relating to the first and/or second monitoring features 130, 140 can be stored locally (e.g. in the local memory of a computer such as a laptop) and then the information streamed or periodically uploaded to the remote location when remote communication is established or available, and the same is true for the test data.

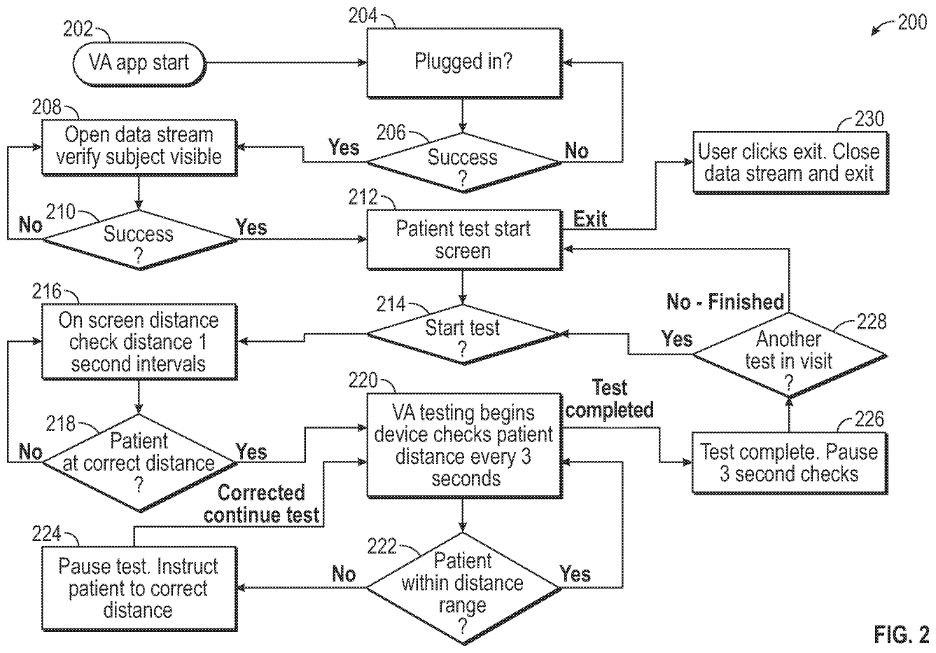

FIG. 2 illustrates a representative flowchart 200 associated with the remote vision test system described above. A visual acuity (VA) application is started at step 202. The application will determine whether or not the system 100 is properly powered or plugged in as indicated in step 204. If the determination in step 204 is “no” then the process can issue a prompt in step 206 (e.g., audible and/or visual prompt) to urge the patient P to check on all connections, and step 204 is then repeated. If the determination in step 206 is “yes”, then the process proceeds to step 208 where a data stream is opened in order to verify that the patient P is detected. For example, if a video recording of the test procedure is desired, integrated camera 150 (FIG. 1) can be enabled and the camera faces in the same direction as the first sensor apparatus 130, i.e., toward the patient P. Alternatively, a separate webcam may be substituted for the integrated camera 150 and the webcam is used to record the patient P during the vision test. The corresponding decision to be made in step 210 (FIG. 2) is either a “no” (that the patient P is not detected) in which case the process returns to step 208 in order to again seek to verify that the patient is detected. If the answer is “yes”, the process continues to the patient test start screen in step 212.

If the choice is made in decision step 214 to proceed with the test, the software proceeds in step 216 to calculate the distance between the monitor 104 and the patient P by enabling the first sensor apparatus 130. For example, the first sensor apparatus 130 may periodically check the distance between the monitor 104 and the patient P at one second intervals, although other intervals may be used without departing from the scope and intent of the present disclosure. Likewise, prompts or cues can be provided to the patient P if the first sensor apparatus 130 determines that the patient is not at the correct distance, or the size of the optotypes may be altered.

Once it has been determined that the patient P is located at the correct distance from the monitor 104, the process moves to step 220 where the visual acuity testing begins. During the visual acuity testing, the first sensor apparatus 130 may also continue to periodically confirm the location of the patient P relative to the monitor 104, for example the interval may be extended to checking the distance every three seconds. Again, the particular interval need not necessarily be three second, and the present disclosure does not preclude other interval lengths.

Once the test is complete, the process moves to step 226 where the process pauses for a predetermined interval or time period, for example a three second pause. If no issue is encountered, in step 228 the patient P may be asked if another test is required. If the answer is yes, then the patient P is directed to step 214 to initiate a new test. If the patient P is finished and no new test is required, then the process moves to step 212 where the patient test screen allows the patient to exit the program as noted in step 230.

Although this preferred process outlines a desired order of individual steps, one skilled in the art will appreciate that still other process steps may be include, some omitted, or possibly another order of one or more steps could be provided.

This written description uses examples to describe the disclosure, including the best mode, and also to enable any person skilled in the art to make and use the disclosure. Other examples that occur to those skilled in the art are intended to be within the scope of the invention if they have structural elements that do not differ from the same concept or that do not differ from the literal language of the claims, or if they include equivalent structural elements with insubstantial differences from the same concept or from the literal language of the claims. Moreover, this disclosure is intended to seek protection for a combination of components and/or steps and a combination of claims as originally presented for examination, as well as seek potential protection for other combinations of components and/or steps and combinations of claims during prosecution.

Although specific advantages have been enumerated above, various embodiments may include some, none, or all of the enumerated advantages. Although exemplary embodiments are illustrated in the figures and description herein, the principles of the present disclosure may be implemented using any number of techniques, whether currently known or not. Moreover, the operations of the systems and apparatuses disclosed herein may be performed by more, fewer, or other components, and the methods described herein may include more, fewer, or other steps. Additionally, steps may be performed in any suitable order.

To aid the Patent Office and any readers of this application and any resulting patent in interpreting the claims appended hereto, applicants do not intend any of the appended claims or claim elements to invoke 35 USC 112 (f) unless the words “means for” or “step for” are explicitly used in the particular claim.

We claim:

1. A vision testing system that tests the vision of an associated patient, the vision testing system comprising:
a display having a first surface for displaying a vision test thereon;
a processing unit operatively associated with the display that selectively displays optotypes on the first surface; and
a monitoring system that senses a distance between the associated patient and the display, and that operationally interfaces with the processing unit to provide data representative of the distance of the associated patient from the display.

2. The vision testing system of claim 1 wherein the monitoring system includes an emitter that sends a signal toward the associated patient where the associated patient is at a location spaced from the display, and a receiver that receives a return signal indicative of the distance from the display.

3. The vision testing system of claim 1 wherein the processing unit includes software for altering a size of the optotypes on the display in response to the data received from the monitoring system.

4. The vision testing system of claim 3 wherein the monitoring system includes one of a light/laser, ultrasonic, or a sonar-type device that emits a signal from adjacent the display toward the associated patient and a receiver that receives a return signal reflected off the associated patient.

5. The vision testing system of claim 4 wherein the monitoring system includes one of an audible or visual cue that directs the associated patient toward or away from the display.

6. The vision testing system of claim 1 wherein the monitoring system includes one of a light/laser, ultrasonic, or a sonar-type device that emits a signal from adjacent the display toward the associated patient and a receiver that receives a return signal reflected by the associated patient.

7. The vision testing system of claim 6 wherein the monitoring system includes one of an audible or visual cue that directs the associated patient toward or away from the display based on a desired distance for the vision test.

8. The vision testing system of claim 1 further comprising a second sensor for detecting ambient light near the display, and the sensor operatively communicating with the processing unit for altering at least one of an ambient light source or the display in response to the detected ambient light.

9. The vision testing system of claim 8 wherein the processing unit controls a brightness level of the display in response to the detected ambient light.

10. The vision testing system of claim 1 further comprising a communications link for uploading vision test results data from a completed test to a remote data storage wherein the data includes at least one of (i) test date, (ii) test time, (iii) patient name and particulars, (iv) testing parameters including detected ambient light level and display brightness, patient distance from display, and displayed optotype size.

11. The vision testing system of claim 1 further comprising a camera for visually recording the patient during the vision testing system.

12. A process of vision testing an associated patient with a vision testing system that includes a vision testing system that includes a display, a processing unit, and a monitoring system operatively interconnected with one another, the testing process comprising:
sensing a distance between the associated patient and the display;
providing data to the processing unit representative of the sensed distance of the associated patient from the display to the processing unit; and
at least one of (I) providing a prompt or cue or (ii) sizing optotypes of the vision test on the display via the processing unit in response to the sensed distance between the associated patient and the display input thereto.

13. The process of claim 12 wherein the sensing step includes emitting a signal from an emitter toward the associated patient, and receiving a reflected signal from the associated patient with a receiver.

14. The process of claim 13 wherein the sensing step includes locating the emitter and the receiver adjacent the display.

15. The process of claim 14 further comprising providing a cue to the associated patient based on the sensed distance of the associated patient from the display.

16. The process of claim 15 wherein the providing step is one of a visual cue on the display or an audible signal.

17. The process of claim 12 further comprising sensing ambient light conditions adjacent the display and altering brightness of the display in response thereto.

18. A process of vision testing an associated patient with a vision testing system that includes a vision testing system that includes a display, a processing unit, and a monitoring system operatively interconnected with one another, the testing process comprising:
sensing a distance between the associated patient and the display;
providing data representative of the sensed distance of the associated patient from the display to the processing unit; and
providing a cue to the associated patient to increase or decrease the distance between the associated patient and the display.

19. The process of claim 18 further comprising sizing optotypes of the vision test on the display via the processing unit in response to the sensed distance between the associated patient and the display input thereto.

20. The process of claim 18 further comprising sensing ambient light conditions adjacent the display and altering at least one of an ambient light source or brightness of the display in response thereto.

21. The process of claim 18 further comprising providing a communications link for uploading vision test results data from a completed test to a remote data storage wherein the data includes at least one of (i) test date, (ii) test time, (iii) patient name and particulars, (iv) testing parameters including detected ambient light level and display brightness, patient distance from display, and displayed optotype size.

22. The process of claim 18 further comprising visually recording the patient during the vision testing system.
